(19) United States
(12) Patent Application Publication
(10) Pub. No.: US 2005/0148874 A1
(43) Pub. Date: Jul. 7, 2005

(54) Ultrasonic imaging aberration correction with microbeamforming

(76) Inventors: George A. Brock-Fisher, Andover, MA (US); Bernard J. Savord, Andover, MA (US)

Correspondence Address: PHILIPS INTELLECTUAL PROPERTY & STANDARDS, P.O. BOX 3001, BRIARCLIFF MANOR, NY, 10510, US

(21) Appl. No.: 11/012,018

(22) Filed: Dec. 14, 2004

Related U.S. Application Data
(60) Provisional application No. 60/531,518, filed on Dec. 19, 2003.

(51) Int. Cl. A61B 8/06

(52) U.S. Cl. 600/447

(57) ABSTRACT

The present invention combines the benefits of aberration correction with the benefits of microbeamforming in an ultrasound diagnostic imaging system, such that both partial beamforming (in the microbeamformer) and at least some part of the phase aberration detection correction processes are accomplished in the transducer probe proximate the transducer array. Accordingly, aberration correction applied at or proximate the microbeamformer in the transducer probe results in a significant simplification of the overall aberration correction technique for the entire ultrasound diagnostic imaging system.

### CROSS REFERENCE TO RELATED CASES

Applicants claim the benefit of U.S. Provisional Application Ser. No. 60/531,518, filed Dec. 19, 2003, which is a continuation-in-part of U.S. Pat. No. 6,682,487.

This invention relates to ultrasonic diagnostic imaging systems and, in particular, to ultrasonic diagnostic imaging systems with aberration correction applied to a microbeamforming operation.

Phased array ultrasonic imaging systems are used to generate real-time images. Such systems include a multiple channel transmit beamformer and a multiple channel receive beamformer coupled to an ultrasonic transducer array or matrix comprising ultrasonic transducer elements. The transmit beamformer generates timed electrical pulses and applies them to the individual transducer elements in a predetermined timing sequence. The transducers respond to the pulses and emit corresponding pressure waves, phased to form a transmit beam that propagates in a predetermined direction from the transducer array.

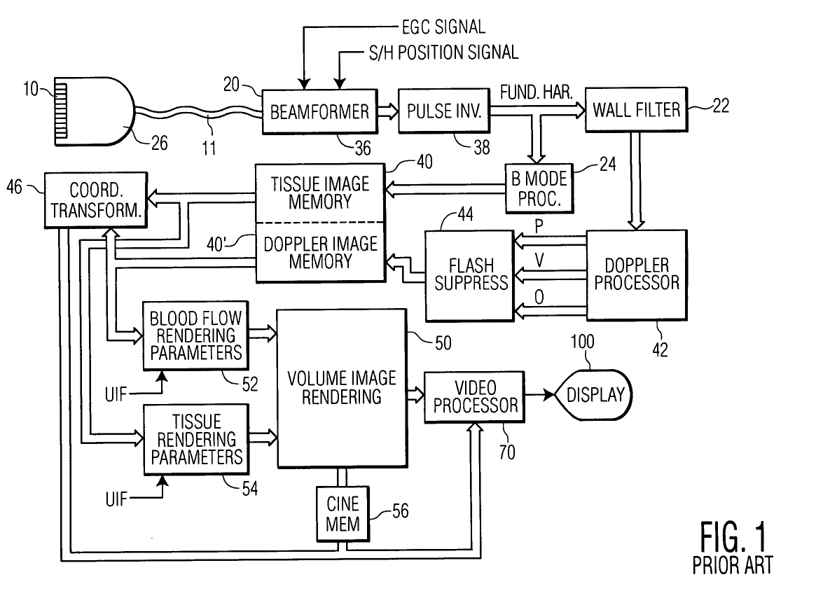

FIG. 1 illustrates a conventional ultrasonic diagnostic imaging system, as taught by commonly owned pending application U.S. Pat. No. 6,497,663, incorporated herein by reference. A scanhead 26 (also referred to interchangeably as a transducer or transducer probe) includes a transducer array 10 connected by a cable 11 to a beamformer 36. The beamformer controls the timing of actuation signals applied to the elements of the transducer array for the transmission of steered and focused transmit beams, and appropriately delays and combines signals received from the transducer elements to form coherent echo signals along the scanlines delineated by the transmit beams. The beamformer is responsive to a scanhead position signal when the transducer array (in the scanhead or probe) is being mechanically moved to sweep ultrasonic beams over a volumetric region, thereby enabling beams to be transmitted when the transducer is properly oriented with respect to the volumetric region.

The output of the beamformer is coupled to a pulse inversion processor 38 for the separation of fundamental and harmonic frequency signals. Fundamental and/or harmonic signals may be B mode processed or Doppler processed, depending upon the desired information to be displayed. For Doppler processing, the signals are coupled to a wall filter 22 which can distinguish between flow, stationary tissue, and moving tissue. The filtered signals are applied to a Doppler processor 42, which produces Doppler power, velocity, or variance estimation. A flash suppressor 44 removes artifacts from scanhead motion, which can contaminate Doppler imaging. The processed Doppler signals are stored in a Doppler image memory 40′.

Signals, which are to be B mode processed, are applied to a B mode processor 24, which detects the signal amplitude. B mode processed signals are stored in a tissue image memory 40. The B mode and Doppler signals are applied to a coordinate transformation processor 46. For conventional two dimensional imaging, the coordinate transformation processor functions as a scan converter, converting polar coordinates to Cartesian coordinates as necessary and filling spaces between received lines with interpolated image data. The scan-converted images are coupled to a video processor 70, which puts the image information into a video format for display on a display 100. The images also may be coupled to a Cineloop® memory 56 for storage in a loop if that function is invoked by the user.

The coordinate transformation processor 46 may be used to scan convert the tissue and Doppler signals in planes of image information over the scanned volume, or transform the coordinates of the image data into a three dimensional data matrix. The coordinate transformation processor typically operates in cooperation with a volume rendering processor 50, which can render a three dimensional presentation of the image data. Three-dimensional images of tissue are rendered in accordance with tissue rendering parameters 54, which are selected by the user through a control panel or user interface (UIF), not shown in the drawing figure.

Three-dimensional images of Doppler information may be rendered in accordance with blood flow rendering parameters 52. These parameters control aspects of the rendering process such as the degree of transparency of tissue in the three-dimensional image, so that the viewer can see the vasculature inside the tissue. Three-dimensional images can be stored in the Cineloop® memory 56 and replayed to display the scanned volume in a dynamic parallax presentation, for instance. To collect the imaging data, the transmit beamformer directs the transducer array to emit ultrasound beams along multiple transmit scan lines distributed over a desired scan pattern. For each transmit beam, the receive beamformer synthesizes one or several receive beams with selected orientations. In 3D ultrasonic imaging, sophisticated conventional systems use a 2D transducer array. Such 2D arrays may have several hundred to several thousand transducer elements, leading to connection challenges.

Each transducer element receives and converts the returned pressure pulses into electrical signals, wherein each electrical signal is processed in a particular (single) processing channel of the receive beamformer. The receive beamformer has a plurality of processing channels with compensating delay elements connected to a summing element. The system selects a delay value for each channel to collect data (returned energy) from a selected point. When the delayed signals are summed whereby a strong signal is realized from signal corresponding to that point. However, signals coming in from other points in the imaging volume have a tendency to destructively interfere.

As the number of transducer elements increases, the number of hard (cable or wire) connecting the elements to receive beamformer (processing) channels also increases. In the near past, microbeamforming technology has developed which provides a microbeamforming solution to the problem of interconnecting the elements of a large array to the transmit and receive beamformers. For example, a conventional 2D array transducer 10″ is shown in FIG. 2, which may be used to implement microbeamforming. To alleviate the interconnection problem, the FIG. 2 solution includes a cable connection and a partitioning of the beamformer between the ultrasound probe or scanhead and the ultrasound system. The details of such a solution may be found in copending and commonly owned U.S. Pat. No. 6,102,863, incorporated by reference herein, and described slightly differently in commonly owned U.S. Pat. No. 5,997,479.

The microbeamforming construction of FIG. 2 performs elevation beamforming in the scanhead 26 and azimuth beamforming in the ultrasound system 101. To better understand the problem, the reader must suppose that the two dimensional array has 128 columns of elements extending in the azimuth direction (indicated by the AZ arrow in the drawing) and six rows of elements in the elevation direction (indicated by the EL arrow). If each element of the array were connected by its own conductor to the ultrasound system, a cable of 768 signal conductors would be required. But as seen in FIG. 2, each column of six elements is coupled to an elevation beamformer 36a which appropriately excites (on transmit) and delays and combines (on receive) signals from the six elements of the column. The six signals in each column essentially operate, therefore, as one elevation beamformed signal. Such elevation beamformed signal is then coupled over a cable conductor to the ultrasound system, where the elevation beamformed signals are beamformed in the azimuth direction. As should be apparent to those skilled in the art, the 128 elevation beamformed signals are coupled over the 128 conductors of a cable 11, realizing a significant reduction in cable size as compared to a probe without scanhead beamforming. At least elevation steering is performed in the elevation beamformer 36a, and preferably both steering and focusing are performed in the elevation beamformer.

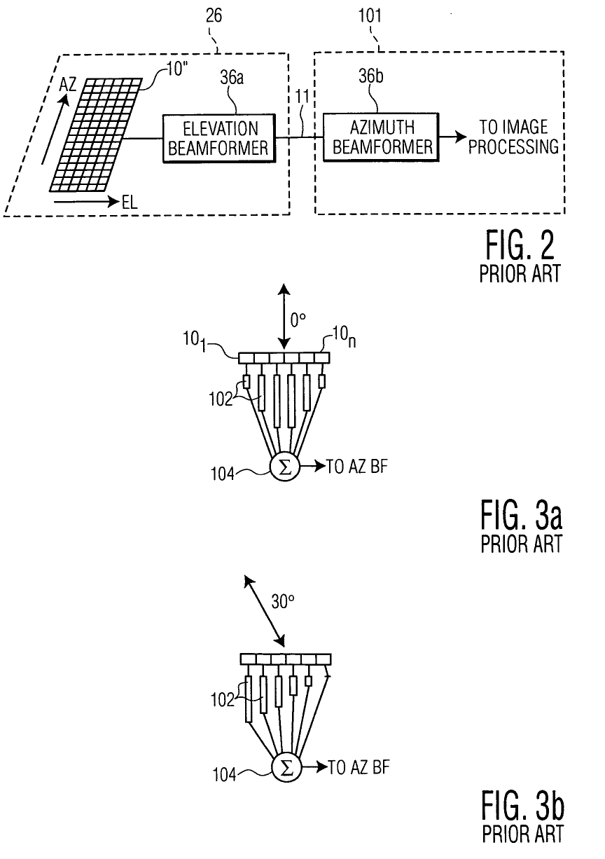

A more detailed operation of such an elevation beamformer is illustrated in FIGS. 3a and 3b. In FIG. 3a, a beam is steered normal to the array transducer as indicated by the 0° arrow extending from the elements 10₁ through 10ₙ, which comprise a column of elements in the elevation direction. Signals at the center of the column are delayed more than signals at the ends of the column as indicated by the relative length of the delays 102 for the different elements to effect a focus. Delayed receive signals are combined by a summer 104, then coupled over a signal lead in the cable 11 to the azimuth beamformer 36b. FIG. 3b illustrates the situation when a beam is to be transmitted or received from the left at a 30° inclination in elevation as indicated by the 30° arrow. In this case signals on the left side of the array are more greatly delayed as indicated by the relative length of the delays 102. Received signals are combined by the summer 104 and coupled through the cable to the azimuth beamformer 36b.

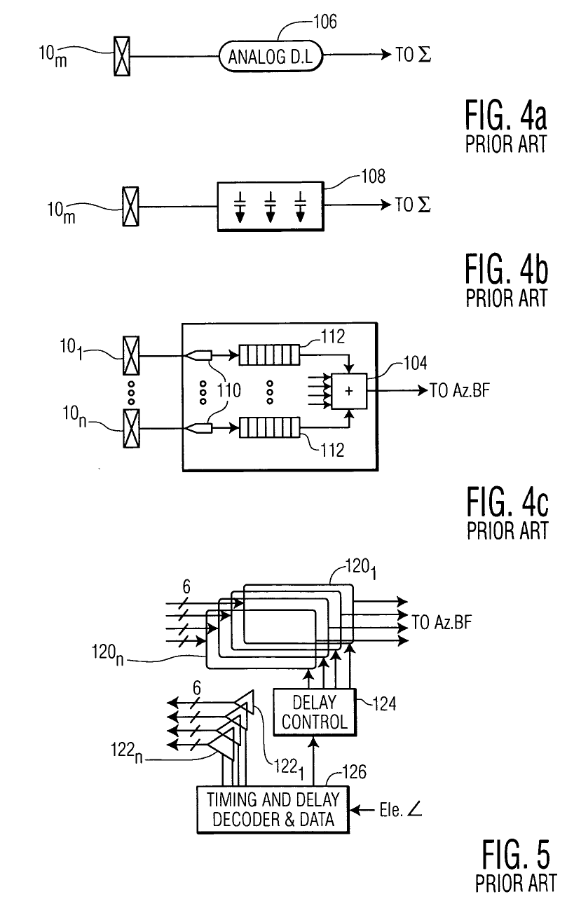

FIG. 4a illustrates an analog implementation of an elevation beamformer, in which each transducer element 10ₘ is coupled to an analog delay line 106. The length of the delay is set by choosing the input or output tap of the delay line and the delayed signals are coupled to an analog summer or to an A/D converter if the signals are to be digitally combined.

FIG. 4b illustrates a beamformer construction where each transducer element 10ₘ is coupled to a CCD delay line 108. The length of the delay is set by choosing an input or output tap that determines the number of charge storage elements in the delay line or by varying the rate at which the charge samples are passed through the charge storage elements. The outputs of the delay lines are summed either in sampled analog format or after being digitized.

FIG. 4c illustrates a digital embodiment of an elevation beamformer. In this example the elevation beamformer has 128 sub-beamformers 120, each processing the signals from one elevation column of six transducer elements. Each of the transducer elements 10₁ through 10ₙ is coupled to an A/D converter 110 and the digitized signals are delayed by a digital delay line 112, which may be formed by a shift register, FIFO register, or random access memory. The appropriately delayed signals are combined in a summer 104 and coupled over cable conductors to the azimuth beamformer. To conserve cable conductors when using multibit signal samples, the data values from several of the beamformer channels 120 can be interleaved (time multiplexed) and sent over the same group of conductors at a data rate sufficient for the desired level of real-time imaging performance.

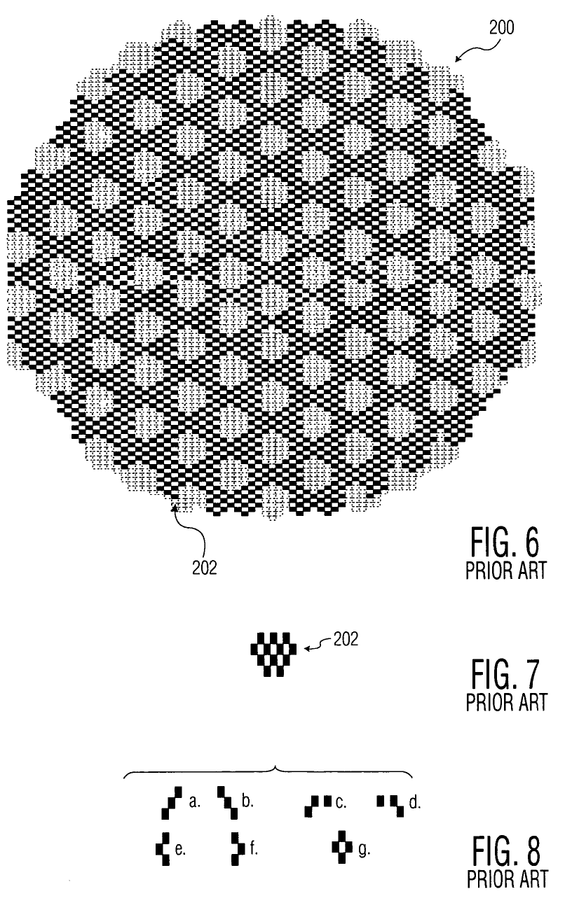

FIG. 5 illustrates the organization and control of a number of beamformer channels 120 of a scanhead elevation beamformer. The beamformer comprises N elevation sub-beamformers 120₁-120ₙ where each sub-beamformer receives signals from a column of transducer elements in the elevation direction, as indicated by the number 6 for this example. Data to control the elevation beamforming (such as elevation angle and focusing) is sent to a timing & delay decoder & data store 126 in the scanhead 26, preferably serially over a cable conductor. This control data is decoded and delay values coupled to a delay control 124, which sets the beamformer channels for the desired delays for each transducer element. For dynamic focusing the delays are changed as echoes are received. The elevation aperture can be varied by applying zero weights to some of the outermost channels when a smaller (near field) aperture is desired.

The data received by the timing & delay decoder & data store 126 is also used to control transmit timing by pulse transmitters 122₁-122ₙ, each of which controls the transmission of the six transducer elements in an elevation column in this example. When received echo signals are processed in the analog domain as illustrated by FIGS. 4a and 4b, the signals from the 128 channels of the elevation beamformer in this example are sent over 128 cable conductors to the azimuth beamformer 36b. When the echo signals are processed digitally the signals from the 128 channels are interleaved (time multiplexed) and sent over digital conductors of the cable 11 to the azimuth beamformer in the ultrasound system 101.

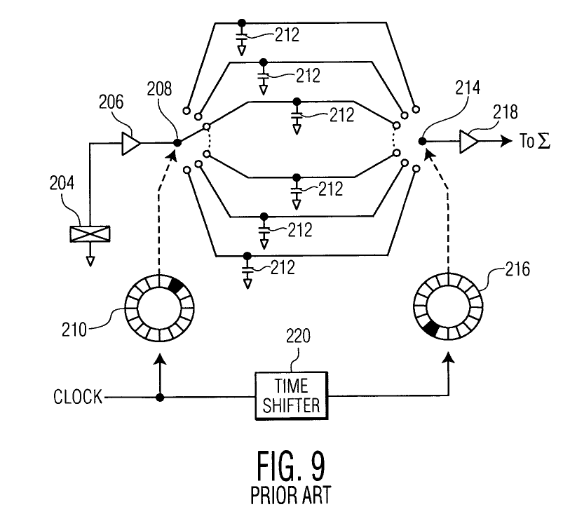

A true 2D electronically steered transducer is illustrated starting with FIG. 6. This drawing shows a plan view of a 2D transducer array 200 of greater than three thousand transducer elements. For ease of illustration the small boxes in the drawing, which represent individual transducer elements, are shown spaced apart from each other. However, in a constructed embodiment, the individual transducer elements are close packed in a repeating hexagonal pattern. The 2D array has an overall dodecahedral outline. The array 200 is seen to be drawn in alternate light and dark groupings 202 of twelve transducer elements.

In one mode of operation, beams are transmitted outward from the center of the array and can be steered and focused in a cone of at least ±30° about a line normal to the center of the array. When steered straight ahead, echoes received from along a transmitted scanline are initially received at the center of the array. Later in time, echoes are received in circular or arcuate groupings of elements centered on and extending outward along the projection of the scanline onto the surface of the array. In the example shown, approximately the central one-quarter of the elements is used for beam transmission. The entire array is available for echo reception.

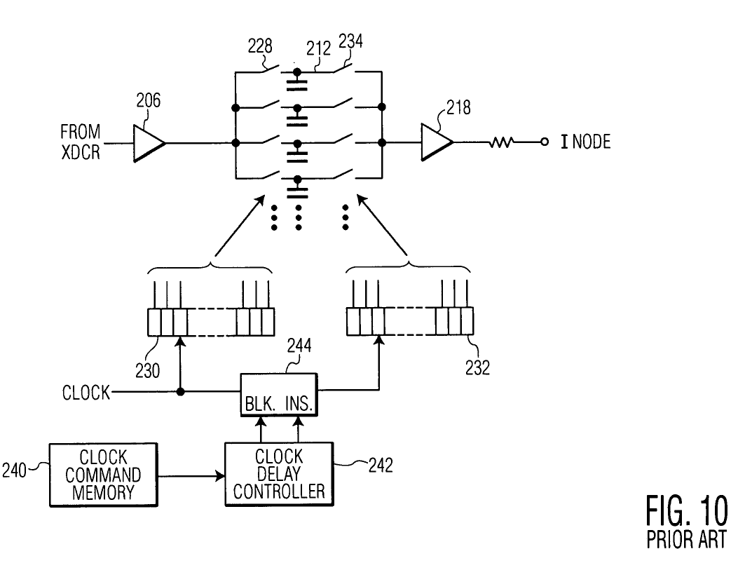

One of the groupings 202 of array 200 (referred to herein as a “patch” of transducer elements) is shown in a separate enlarged view in FIG. 7. These irregular hexagonal patches 202 of twelve elements are beamformed together during echo reception as discussed in detail below. Elements in the center of the array (approximately 750 elements) are connected in groups of three for transmission by high voltage mux switches. FIGS. 8a-8f show some of the three-element configurations that are possible during beam transmission. The transmit groupings can also simply be three elements adjacent to each other in a straight line. The exact configuration or configurations used to transmit a given beam depend upon the desired beam characteristics and its azimuth. Four elements may also be connected together for transmission as illustrated by the diamond shaped grouping of four elements in FIG. 8g.

Since a cable with more than three thousand conductors is not currently practical, each patch of twelve elements of the array is beamformed in the scanhead (or transducer probe 26). This reduces the number of signals which must be coupled to the ultrasound system beamformer to approximately 256. Then, a 256 channel beamformer 26 in the ultrasound system can be used to beamform the partially beamformed signals from the scanhead. Because the elements of each receive patch of twelve elements of the 2D array are sufficiently small, contiguously located, and closely packed, the echo signals received by the elements of a patch will be aligned to within one wavelength at the nominal receive frequency for steering angles of approximately 40° or less (neglecting focal delays). The echoes of the elements are then sampled to bring all of the patch element signals into precise time alignment. The sampling is done with a range of sampling delays with a precision of a fraction of a wavelength to bring the signals from all of the patch elements to a time alignment within the precision of the sampling clock quanta, preferably 1/16 of a wavelength or less.

The time-aligned signals from the patch elements are then combined. This beamforming of each patch is done by microelectronics located immediately behind the transducer array in the scanhead to facilitate interconnections (i.e., by the microbeamformer. Sample time shifting and alignment is performed by the sampling delay line shown in FIGS. 9 and 10. Each element 204 of a patch of elements which is to be partially beamformed is coupled by way of an amplifier 206 to a sampling input switch 208. The sampling input switch 208 is continually conducting samples of the transducer signal onto capacitors 212 in a sequential manner. The sequencing of the switch 208 is under control of a ring counter 210, which is incremented by a clock signal. As the darkened segment of the ring illustrates, the sampling input switch is continually sampling the input signal onto successive ones of the capacitors 212 in a circular manner. The amplifier 206 has a bipolar output drive so that the charge of a capacitor can be either increased or decreased (discharged) to the instantaneous signal level at the time of sampling.

The signal samples stored on the capacitors 212 are sampled by a sampling output switch 214, which samples the stored signals in a sequential manner under control of a second ring counter 216. As shown by the darkened segment on the ring of the second ring counter 216, the sampling output switch 214 samples the stored signals in a particular time relationship to the input switch and its ring counter. The time delay between the input and output sampling is set by a time shifter 220, which establishes the time delay between the two ring counters. Thus the time of sampling of the output signal samples can be incrementally advanced or delayed as a function of the timing difference between the two ring counters. This operation can be used to bring the output signal samples of all the elements of a patch into a desired time alignment such as the sampling time of a central element of the patch. When the signals from all of the elements of the patch are within a desired range of sampling time, the signals can be combined into one signal for further beamforming in the ultrasound system. The time aligned output signals are further amplified by an amplifier 218 and coupled to a summer for combining with the signals of the other elements of the patch.

In integrated circuit fabrication, the sampling switches of FIG. 10 do not have rotating wipers as illustratively shown in FIG. 9, but are formed by a plurality of gates 228. Each of the gates 228 is controlled by the output of an output stage of a shift register 230, which is arranged to circulate one bit and thereby operate as a ring counter. When the bit is shifted to a particular stage of the shift register 230, the gate 228 connected to that stage is closed to conduct a signal sample to its capacitor 212. The output switches are similarly constructed as a series of parallel gates 234, and are similarly controlled by stages of circulating shift register 232. Signal samples taken from the capacitors 212 are amplified and resistively coupled to a current summing node for summation with the other signals of the grouping.

A clock command memory 240 is located in the scanhead and preferably on the same integrated circuit as the sampling circuitry. The clock command memory stores data identifying the time delays needed for one or more receive echo sequences. The control data for the current beam is coupled to a clock delay controller 242, which controls the relative time relationship between the two ring counters. The controller 242 does this by blocking clock cycles applied to the first ring counter 230 from reaching the second ring counter 232, or by inserting additional clock cycles into the clock signal. By blocking or inserting shift register clock pulses to the second ring counter the relative timing between the two ring counters is adjustably advanced or retarded. The time aligned samples from all of the transducer elements of the patch are then combined at a current summing node I Node. The summed signals from the patch are coupled through the scanhead cable to the ultrasound system beamformer.

With the addition of a second sampling output switch for each element controlled in a different time relationship than the first sampling output switch, and a second summer for the second sampling output switches of the patch elements, a second, receive beam can be produced at the same time as the first receive beam. Thus, each patch becomes a small multiline receiver receiving two (or more) receive beams simultaneously, which is useful in the multiline embodiment described below.

The microbeamformer for the patches can utilize other architectures such as charge coupled delay lines, mixers, and/or tapped analog delay lines, as should be known to those skilled in the art.

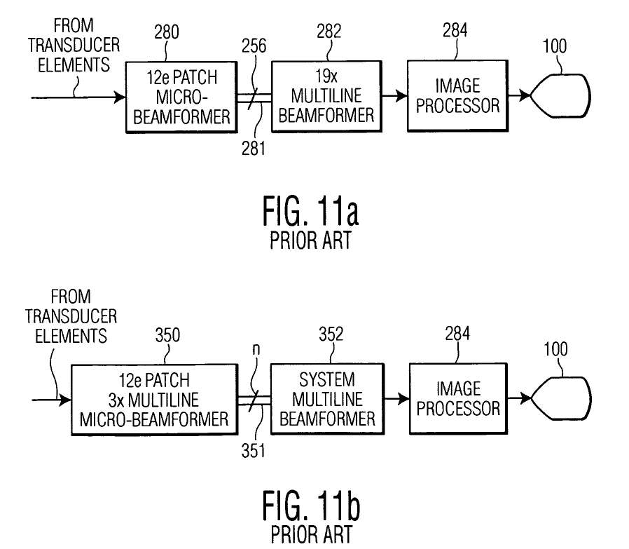

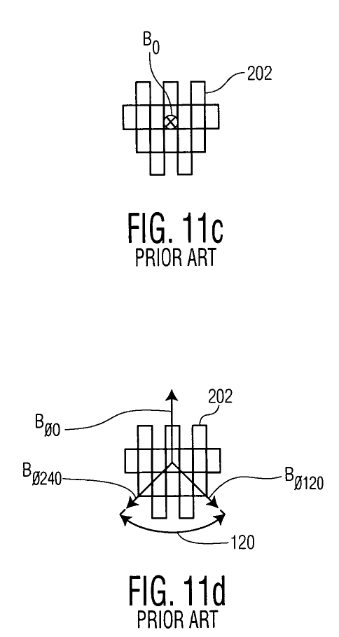

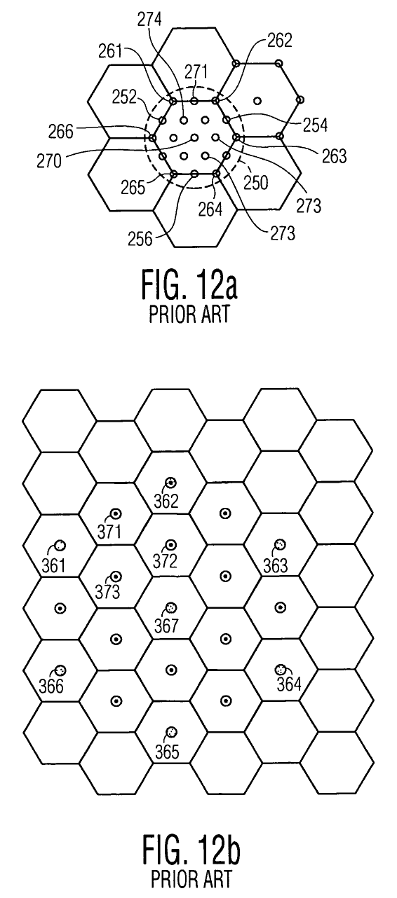

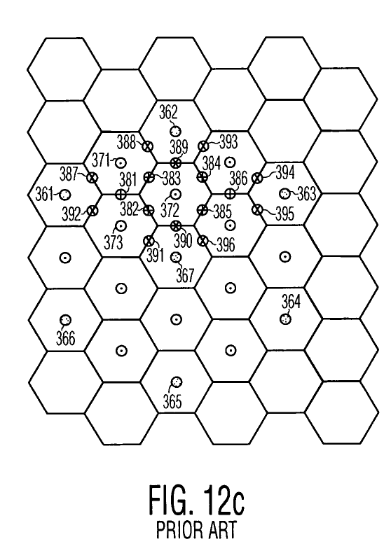

Three dimensional imaging requires that the volumetric region be sufficiently sampled with ultrasound beams over the entire volume. This requires a great many transmit-receive cycles which causes the time needed to acquire a full set of volumetric data to be substantial. The consequences of this substantial acquisition time are that the frame rate of a real-time 3D display will be low and that the images will be subject to motion artifacts. Hence it is desirable to minimize the time required to acquire the necessary scanlines of the volumetric region. A preferred approach to this dilemma is to employ multiline beamforming, scanline interpolation, or both, as shown in FIG. 11 and FIG. 12.

While beams may be steered in a square or rectangular pattern (when viewed in cross-section) to sample the volume being imaged, in a preferred embodiment the beams are oriented in triangular or hexagonal patterns in the volumetric region to sufficiently and uniformly spatially sample the region being imaged. FIG. 12a is a cross-sectional view through the volumetric region in which scanlines in the volumetric region are axially viewed. In this example nineteen scanlines are produced for every transmit beam. The scanline locations are spatially arranged in hexagonal patterns. The nineteen scanline locations of one hexagonal pattern are denoted by circles which represent axial views along the scanlines. The nineteen scanline locations are insonified by a “fat” transmit beam of a desired minimum intensity across the beam.

The transmit beam in this example is centered on the location of scanline 270, and maintains the desired acoustic intensity out to a periphery denoted by the dashed circle 250, which is seen to encompass all nineteen scanline locations. The echoes received by the elements of the transducer array are partially beamformed by a micro-beamformer 280 in the scanhead, as described above, and coupled to a 19× multiline beamformer 282 in the ultrasound system as shown in FIG. 11a. In this example a 2D transducer array of 3072 elements is operated in patches of 12 elements, producing 256 patch signals which are coupled to the ultrasound system by a cable 281 with 256 signal conductors without multiplexing. The 19× multiline beamformer processes the 256 echo signals received from the transducer patches with nineteen sets of delays and summers to simultaneously form the nineteen receive scanlines 252-274 shown in FIG. 12a. The nineteen scanlines are coupled to an image processor 284, which performs some or all of the harmonic separation, B mode, Doppler, and volume rendering functions previously described in FIG. 1. The three dimensional image is then displayed on the display 100.

Interpolation may be used to form scanline data, either alternatively to or in conjunction with multiline scanline formation. FIG. 12b illustrates a series of scanlines 361-367 marked by the darkened circles which have been acquired from a volume being imaged in a hexagonal pattern as indicated by the background grid pattern. The scanlines 361-367 can be acquired individually or in groups of two or more by multiline acquisition. Scanlines at the undarkened circle locations are interpolated from the acquired scanlines using two-point r.f. interpolation. The interpolated scanline 371 is interpolated by weighting each of the adjacent scanlines 361 and 362 by ½, then combining the results. The weights used are a function of the location of the scanline being produced in relation to the locations of the three received scanlines whose values are being interpolated.

Similarly, interpolated scanline 372 is interpolated using adjacent scanlines 362 and 367, and interpolated scanline 373 is interpolated using adjacent scanlines 361 and 367. Each group of three scanlines is used to interpolate three intermediate scanlines using weighting factor, which are a factor of two (2⁻¹), enabling the interpolation to be performed rapidly by shifting and adding the bits of the data being interpolated. This avoids the use of multipliers and multiplication and affords high-speed processing advantageous for real-time 3D display rates.

FIG. 12c illustrates a further iteration of the interpolation of FIG. 12b in which the scanline density of the volume is increased even further by interpolation. In this illustration two further sets of scanlines 381-383 and 387-392 are interpolated between the previous set. These scanlines may be interpolated using the previously interpolated set of scanlines, or they may be interpolated directly (and simultaneously, if desired) from the acquired scanlines 361,362,367. These scanlines also have the advantage of being weighted by weighting factors, which are a factor of two. The set of interpolated scanlines most central to the three received scanlines, 381-383, are interpolated using weighting factors of ½ and ¼. Scanline 381, for instance, is produced by (½(scanline 361)+¼(scanline 362)+¼(scanline 367)). The outer set of scanlines is produced by ¼, ¾ weights as described in U.S. Pat. No. 5,940,123. Scanline 392, for instance, is produced by (¼(scanline 367)+¾(scanline 361)) or, to avoid multiplication, (¼(scanline 367)+¼(scanline 361)+¼(scanline 361)+¼(scanline 361)).

FIG. 12c illustrates corresponding sets of interpolated scanlines for received scanlines 362,363,367, including the central group of scanlines 384-386, and the outer set of scanlines 393-396. To reduce motional artifacts, the received scanline data can be filtered in either r.f. or detected form prior to display.

The above conventional system uses a linear interpolation filter kernel. It is also possible to use an interpolation kernel that has a non-linear shape (such as, for example, cosine, sinc, etc.) However the filter coefficients of these other filters will generally not have the desirable power of two property.

The use of patches to reduce the size of the cable needed to connect the scanhead to the ultrasound system may, under certain operating conditions, give rise to undesired grating lobes in the scanhead's beam pattern. This is due to the grouping of individual transducer elements into a single unit, giving the transducer array a coarser pitch, even with the use of micro-beamforming as described above. This problem can be reduced by considering each patch to be a sub-aperture of the entire 2D array which is capable of receiving signals from multiple, closely spaced scanlines in the transmit beam field.

The signals from the sub-apertures can be delayed and summed to form a group of multiline received scanlines. Grating lobes, which arise by reason of the periodicity of the sub-apertures and can contribute clutter to the final image, are reduced by producing two or more differently steered signals from each sub-aperture (patch). The steering difference is kept small, within the beamwidth of the patch. By keeping the steering delay profile less than λ/2, significant grating lobes are kept out of the image field.

A simple 1D example illustrates these effects. Consider a sixty-four element 1D linear array with inter-element spacing (pitch) of λ/2. The array is divided into four patches of sixteen elements each. Two beams are steered to the left and right of a nominal direction on each patch. The steering angles are limited so that other lines or samples can be interpolated between these two received multilines. It is desirable for the multilines to be radially far enough apart to support the creation of interspaced interpolated lines, but close enough together so that r.f. interpolation will not form artifacts due to spatial undersampling.

For example, if the steering delays are limited to correspond to less than ±λ/8, then the two steered beams from each patch will fall within approximately the −1 dB width of the nominal patch beampattern. Also, because the steering delay between the left and right multiline on any element is thus limited to λ/4, r.f. interpolated lines can be produced using a simple two tap interpolation filter (λ/2 delays would correspond to the Nyquist criterion). The λ/8 delay limitation limits the steering angle to approximately ±(λ/8)/(4*λ) or 1/32 radians. Thus the angle between the left and right multilines can be about 1/16 radians, or about 3.6 degrees. If two other lines are symmetrically interpolated between the two received multilines, the resulting line spacing is approximately 1.2 degrees. A greater number of more closely spaced multilines or interpolated lines can also be produced as desired.

In the 1D array example, instead of producing a single scanline from each patch steered in the nominal steering direction, two scanlines are produced, one steered slightly left of the nominal steering direction and one steered slightly right. In the case of a 2D array, several variations are possible. For a rectilinear 2D array, four scanlines are produced for each patch, steered left, right, up and down in quadrature relationship.

For a triangular-based 2D array such as a hexagonal array, three scanlines are produced at rotations of 120° as shown in FIG. 11d. The scanlines produced in this drawing are identified as B_{φ0}, B_{φ120} and B_{φ240}, respectively, where the subscript number refers to the direction of rotation in the plane normal to the nominal steering direction of the patch and the angle φ is the small angle at which each scanline is tilted from the nominal steering direction. The angle φ is kept small as described above so that the three scanlines are kept within the beamwidth of the nominally steered beam. FIG. 11c illustrates a single scanline B₀ oriented normal to the patch 202, as would be produced by the system shown in FIG. 11a, which has a beam nominally steered normal to the face of the patch 202.

Although the foregoing examples suggest the use of a rectangular scan geometry for a rectilinear array and a triangular scan geometry for a hexagonal array, the scan geometry is not intrinsically linked to array geometry. A rectangular scan can be performed using a hexagonal array and vice versa.

A system operating as illustrated by FIG. 11d is shown in FIG. 11b. The scanhead in this drawing includes a 12-element patch micro-beamformer, which produces three multiline signals from each patch (B_{φ0}, B_{φ120} and B_{φ240}, for example) instead of one line, as did the micro-beamformer 280 of FIG. 11a. The micro-beamformed patch multilines are sent over the n conductors of a cable 351 to the ultrasound system's multiline beamformer 352. The multiline scanlines from all of the patches are combined in the system multiline beamformer 352 to form multiple scanlines. It is also possible to perform r.f. interpolation between the multiline scanlines. However, rather than combine (beamform) the multiline signals from each patch and then perform r.f. interpolation on the beamformed signals, it is preferred that r.f. interpolation is performed on signals received from each patch separately prior to beamforming combination.

In this case, prior to the weighting and summation operations of r.f. interpolation, each patch signal for each nominal steering direction is slightly delayed or advanced by an amount determined by each patch position and the offset of the interpolated line from the nominal line. The effect of the delays is to maximize the coherence of the patch waveforms combined in the r.f. interpolation step. This reduces interpolation errors and improves sensitivity. Specifically, if N interpolated lines are produced from M patches, each patch having K multilines, then MN r.f. interpolators are required with each interpolator preceded by K delay states, one for each multiline.

This same approach (i.e., delay+individual patch r.f. interpolation prior to patch signal combination) can also be used on patch signals received from different directions in a non-multiline mode provided that target motion between successive transmits is not excessive. The multiple scanlines are then processed by the image processor 284 and displayed on the display 100 as described previously. The number n of receive signal conductors of the cable is 768 if three multilines from each of 256 patches are sent simultaneously without multiplexing, a number which can be reduced by multiplexing if desired. The patch multilines received by the ultrasound system can be interpolated to form additional scanlines prior to system beamformation if desired. However, since the processing of interpolation (weighting and summing) is mathematically compatible with that of beamformation, the patch multilines can be supplied directly to the system beamformer for formation of beamformed multilines.

Several display formats may be used for the three dimensional display of the present invention. FIG. 13 shows a volumetric region 300 which is being scanned by a 2D transducer array 200. The volumetric region scanned can be in any desired shape, such as square, cylindrical, or pyramidal, depending upon the steering of the beams from the transducer. In this example the volumetric region 300 is shown as a hexagonal pyramid. Shown within the volumetric region 300 is an image plane 302, which is delineated by the double lines. The image plane 302 is scanned in a time interleaved manner as the volumetric region 300 is scanned. The time interleaving enables the echo data from the image plane 302 to be fully acquired in less time than that required to scan the full volumetric region 300 and the frame rate of display of the image plane 302 is thus greater than that of the volumetric display.

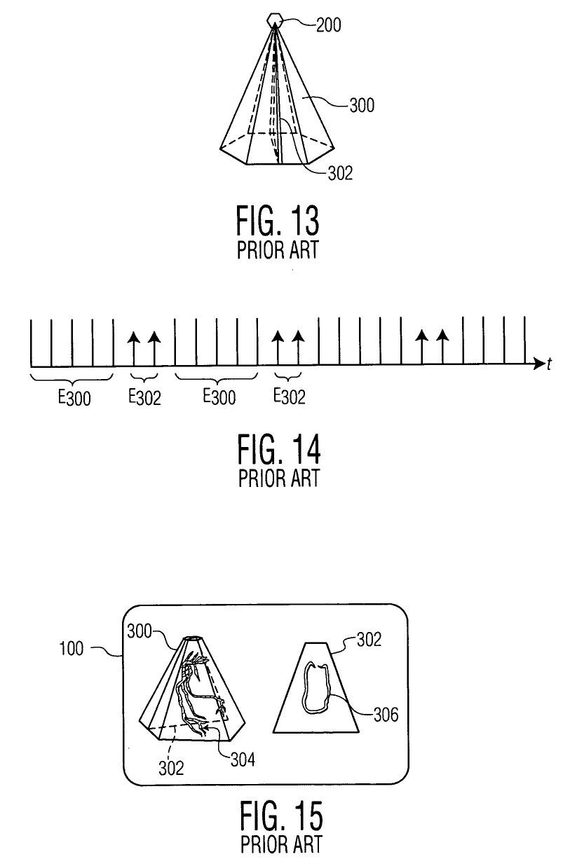

The time interleaving of the volumetric and planar image data is illustrated by FIG. 14. This drawing shows a sequence E₃₀₀ during which echo data is acquired for the volumetric display. This sequence is periodically interrupted during which echo data E₃₀₂ for the planar display is acquired. Some of the planar echo data can be used for both displays. The relative durations of the sequences and the number of transmit-receive cycles needed for each display determine the frame rate relationship of the two displays.

The volumetric and the planar images are preferably displayed together as illustrated in FIG. 15. On the left side of the display 100 is a three dimensional display of the volumetric region 300, which shows the structure 304 in the volumetric region in a three dimensional presentation. On the right side of the display 100 is the two dimensional image plane 302, effectively showing a cut plane 306 through the three dimensional structure 304. While the frame rate of display of the three dimensional image 300 may be relatively low, the frame rate of display of the two dimensional image 302 will be much higher, which is useful when diagnosing moving objects such as the heart. The location of the two dimensional plane 304 is typically indicated in the three-dimensional display, as shown in this example. This gives the user a basis of reference for the two dimensional image plane within the volumetric region.

The user has the ability to move the location of the cut plane 306 within the volumetric region so that a selected pathology can be viewed at the higher frame rate. By manipulating a pointing device, such as a mouse or trackball, the position of the image plane 302 within the volumetric region 300 can be changed on the left side of the display. The user is given a choice of rotating the cut plane about the center axis of the volumetric region, or of dragging or moving the cut plane to an arbitrarily chosen position within the volume. Thus, the display 100 displays a volumetric region at a relatively low frame rate, and a selected plane at a higher real-time frame rate. This method applies when the cut plane extends from the transducer aperture, that is, the cut plane is not a “c” plane.

The present inventors have recognized a need for applying aberration correction to the sub-array outputs of a sub-array beamformer, for example, to a matrix transducer. That is, the inventors hereof have recognized the need for applying sub-array correction to sub-array beamformers in a arrays with a plurality of dimensions, thereby performing the aberration correction in a manner which corresponds to microbeamforming, whereby the beamforming and aberration correction operations, and the hardware needed to process arrays of very large numbers of elements, especially matrix arrays used for 3-D imaging are simplified.

FIG. 1 is a diagram illustrating a conventional ultrasonic diagnostic imaging system;

FIG. 2 is a diagram illustrating the partitioning of beamforming between a scanhead and an ultrasound system;

FIGS. 3a and 3b are diagrams illustrating the steering of a beam in the elevation direction by a scanhead beamformer;

FIGS. 4a, 4b and 4c are diagrams illustrating different embodiments of a scanhead elevation beamformer;

FIG. 5 is a diagram illustrating an azimuth beamformer, which operates with the elevation beamformers of FIGS. 4a, 4b and 4c;

FIG. 6 is a plan view of a conventional two dimensional transducer array for three-dimensional scanning;

FIG. 7 is a diagram illustrating a receive sub-aperture of the transducer array of FIG. 6;

FIGS. 8a-8g illustrate different transmit sub-apertures of the transducer array of FIG. 6;

FIG. 9 is a diagram illustrating scanhead microcircuitry for sampling the signals received by a transducer element of the transducer array of FIG. 6 in a desired time relationship;

FIG. 10 is a more detailed view of the microcircuitry of FIG. 9;

FIG. 11a illustrates a scanhead micro-beamformer and multiline beamformer system suitable for processing the signals received by the transducer array of FIG. 6;

FIG. 12a illustrates operation of the system of FIG. 11a for a hexagonal scanning pattern;

FIGS. 12b and 12c illustrate the use of interpolation to develop a hexagonal scanline pattern;

FIG. 11b illustrates the use of a multiline scanhead micro-beamformer in combination with a system multiline beamformer;

FIGS. 11c and 11d illustrate single line and multiline beam steering from a 2D transducer array patch;

FIG. 13 illustrates a three dimensional volume containing a two dimensional image plane;

FIG. 14 illustrates the time interleaved sampling of the three dimensional volume and two-dimensional image plane of FIG. 13;

FIG. 15 illustrates a duplex display of the three dimensional volume and two-dimensional image plane of FIG. 13;

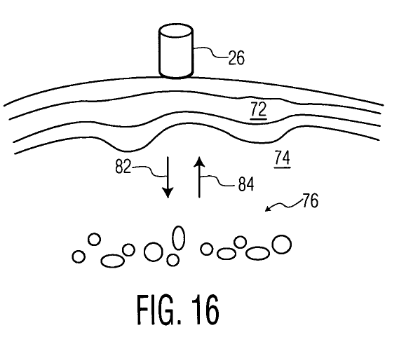

FIG. 16 illustrates an imaging scenario in which the wave fronts encounter aberrators as they travel through the body;

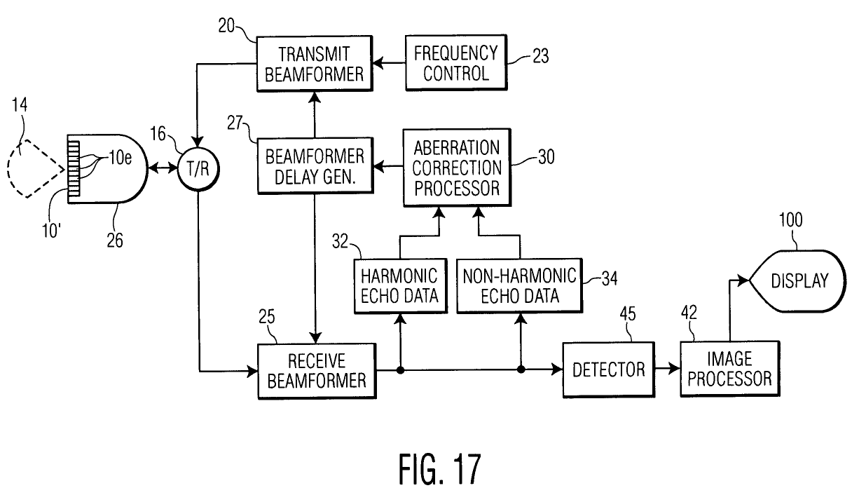

FIG. 17 illustrates an ultrasonic diagnostic imaging system with aberration correction;

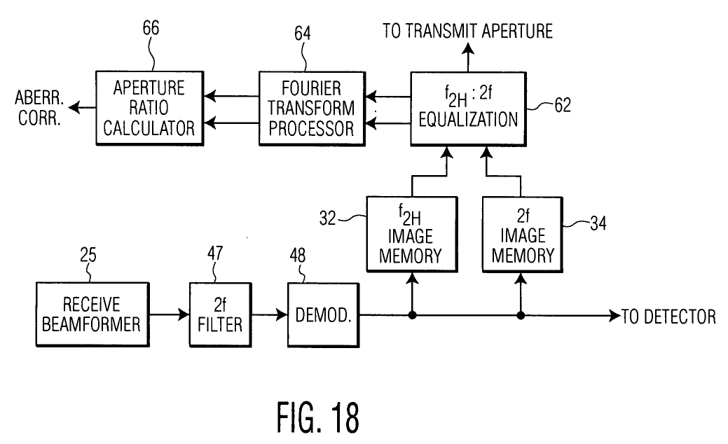

FIG. 18 illustrates details of the aberration correction subsystem of the ultrasonic imaging system of FIG. 17;

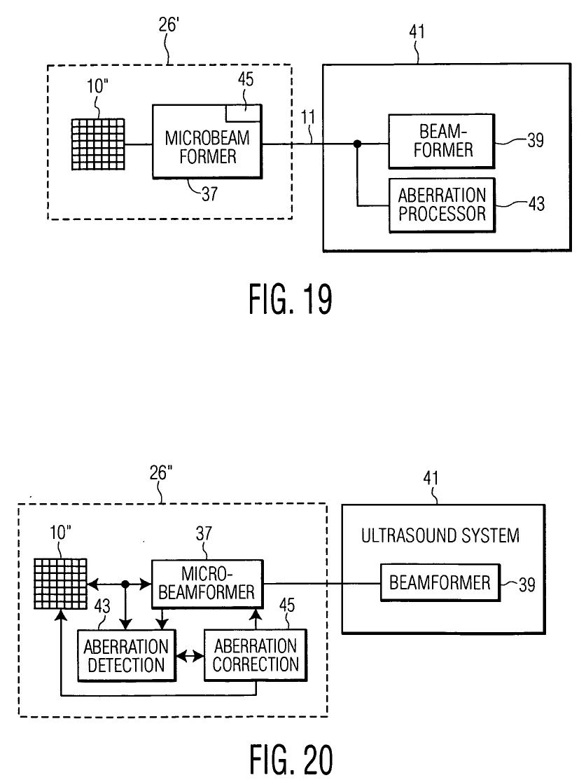

FIG. 19 is a diagram showing one embodiment of an ultrasound system of this invention; and

FIG. 20 is a diagram of another embodiment of the invention.

Reference will now be made in detail to the preferred embodiments of the present invention, examples of which are illustrated in the accompanying drawings, wherein like reference numerals refer to like elements throughout.

The present invention utilizes aberration correction, for example, phase aberration correction, within a microbeamforming operation, particularly within ultrasound systems which utilize matrix arrays. The detailed description which follows discusses first aberration correction, in at least one implementation known to those skilled in the art, and then describes in detail how the inventors have improved on the art of aberration correction and microbeamforming to realize the present inventions. The text and drawings present the invention(s) in terms of routines and symbolic representations of operations of data bits within a memory, associated processors, and possible networks, and network devices. These descriptions and representations are the means used by those skilled in the art to effectively convey the substance of their work to others skilled in the art. A routine is here, and generally, conceived to be a self-consistent sequence of steps or actions leading to a desired result. Thus the term “routine” is generally used to refer to a series of operations performed by a processor, be it a central processing unit of an ultrasound system, and as such, encompasses such terms of art as “program,” “objects,” “functions,” “subroutines”, and “procedures.”

In general, the sequence of steps in the routines require physical manipulation of physical quantities. Usually, though not necessarily, these quantities take the form of electrical or magnetic signals capable of being stored, transferred, combined, compared or otherwise manipulated. Those of ordinary skill in the art conveniently refer to these signals as “bits,” “values,” “elements,” “symbols,” “characters,” “images,” “terms,” “numbers” or the like. It should be recognized that these and similar terms are to be associated with the appropriate physical quantities and are merely convenient labels applied to these quantities. As used herein, the routines and operations are machine operations to be performed in conjunction with human operators. Useful machines for performing the operations of the present invention(s) include Philips' SONOS ultrasound systems, and other similar devices. In general, the present invention relates to method steps, software, and associated hardware including computer readable medium, configured to store and/or process electrical or other physical signals to generate other desired physical signals.

The apparatus as taught hereby is preferably constructed for the required purpose, i.e., ultrasound imaging, but the methods recited herein may operate on a general purpose computer or other network device selectively activated or reconfigured by a routine stored in the computer and interface with the necessary ultrasound imaging equipment. The procedures presented herein are not inherently related to any particular ultrasound system, computer or other apparatus. In particular, various machines may be used with routines in accordance with the teachings herein, or it may prove more convenient to construct more specialized apparatus to perform the required method steps. In certain circumstances, when it is desirable that a piece of hardware possess certain characteristics, these characteristics are described more fully in the following text. The required structure for a variety of these machines may appear in the description below.

As mentioned briefly above, while the microbeamforming time-delay beam steering and focusing techniques discussed do provide for high-quality real-time diagnostic ultrasound imaging, there are still several limiting factors on image quality. For example, phase aberration which occurs due to varying propagation conditions for ultrasound signals as they propagate from individual transducer elements through intervening tissue layers degrades image quality. As a result, an exact focus of transmitted and received beams may not always occur. In theory, many of these effects can be overcome by adjustment of the delays used to focus the transmitted and received beams. Various techniques, known as aberration correction or adaptive beamforming have developed in an effort to improve images. That is, various techniques have developed which include correcting time delays and/or amplitude and/or phase error of signals emitted or received at individual transducer elements in order to account for phase aberration.

Determining the delay adjustments needed, and doing so adaptively and in real time, have been the subject of investigations for many years. One approach to adjusting for phase aberration in ultrasound imaging involves cross correlating echoes received by neighboring transducer elements or groups of elements to estimate arrival time differences. Another varies focusing delays so as to maximize the brightness of speckle or reflectors in the image field. While many of these efforts have tried to provide compensation for time shifts in the receiving aperture, others have looked to provide time-shift compensation for the transmit aperture. These approaches are typically iterative in nature, and face the problems of converging to an acceptable result with an infinitely variable and at times moving combination of reflectors and aberrators in the image field.

U.S. Pat. No. 6,682,487, commonly-owned and incorporated herein by reference, provides a structure and method by which aberration corrections are computed by comparing harmonic and non-harmonic images to derive aberration correction estimates. In one embodiment, the harmonic image is a reference image against which aberrations in the non-harmonic image are compared. A preferred acquisition technique therein includes transmitting at a frequency f and receive at a frequency n*f to acquire the harmonic image and transmitting at a frequency n*f and receive at a frequency n*f to acquire the non-harmonic image.

The aberration correction estimates are produced by back-propagating the image data to find the aperture correction data for the two images, particularly useful for aberration correction of data from a two dimensional array which is divided into N×M subarrays. FIG. 16 (similar to FIG. 1 of U.S. Pat. No. 6,682,487) shows an ultrasound transducer probe 26, including an array of transducer elements (not shown in the figure) depicted imaging body tissues which include scatterers 76. These scatterers may be within the tissue of an organ to be imaged, such as the heart.

Ultrasound waves, transmitted (as indicated by arrow 82) and received (as indicated by arrow 84) as steered and focused beams, are emitted and received by the transducer probe 26. As the wave fronts travel into the body they initially encounter non-homogeneous tissue 72, e.g., skin, fat, muscle, bone, etc. The wave fronts may travel at a slightly different velocities through this non-homogeneous tissue than the velocity assumed by the geometric focus equations of the ultrasound system. Different wave fronts from an array transducer, which take slightly different paths to the scatterers 76, can arrive at the scatterers at slightly different times rather than simultaneously at the focal point as intended.

The non-homogeneous tissues are also encountered by the echo wave fronts on their return to the elements of a transducer array. The aberrators cause less than optimal focusing of the received signals, as the delays computed by the geometric focus equations to bring the signals from the elements of the array into time coherence do not take the time shifts caused by the aberrators into account.

When an ultrasound wave is transmitted by the transducer probe 26 at a frequency f and harmonic echo signals at a frequency n*f are received. In the case where n=2, as the waves at the fundamental frequency f pass through the non-homogeneous tissue 72 they are affected by the aberrators in the tissue and time-shifted in relation to the aberrators encountered. However, the resulting phase distortion is relatively small by reason of the relatively low frequency f of the transmit waves. The waves then pass through soft tissue 74 on their paths to the scatterers 76 and by doing so the non-linear media of the soft tissue distorts the waves, causing harmonic signal components at the frequency 2f to develop.

Harmonic development increases as the pressure wave comes into focus in the region being imaged. Since the majority of harmonic development occurs after passage of the waves through the non-homogeneous media, the harmonic wave components are relatively unaffected by the aberration effects of this tissue. The harmonic signals are reflected by the scatterers 76 and the echoes return to the transducer probe 26 for reception.

The transducer probe also transmits and receives fundamental frequency signals. In the preferred embodiment the fundamental frequency signals are transmitted at a frequency of nf and received at the frequency nf. In this example where n=2, the transmitted frequency is 2f. As the waves at this frequency travel through the non-homogeneous media 72, they are affected by the time-shifts caused by the aberrators of the non-homogeneous tissue which cause relatively significant phase shifts at the higher frequency of 2f. The fundamental frequency waves 2f also do not enjoy the benefit of developing after passage through the non-homogeneous tissue as the harmonic signals did. The fundamental frequency signals arrive at the scatterers 76 and are reflected back to and received by the transducer probe 26.

In the absence of aberration effects the harmonic and non-harmonic received signals, both at a frequency of 2f, should be substantially identical. As discussed below, this will be substantially the case when there is no intervening motion between transmit events and the round trip spectral response of the signals is equalized by considerations such as the relative transmit power or gain compensation applied to the fundamental and harmonic echoes (B/A). But in the presence of aberration the non-harmonic signals should be significantly more degraded with respect to the harmonic signals for the reasons given above. Thus the harmonic signals are used as a baseline or reference against which aberration effects in the non-harmonic signals are compared and aberration corrections made.

The harmonic and non-harmonic signals are compared by the process of back propagation. The echo data of both types of signals is back-propagated to determine the aperture data for the two signal types and a complex ratio of the aperture data is formed to produce an estimated aberration correction. The aberration correction values are combined with the geometric focus delay values (or the geometric focus delay values as modified by previous aberration correction) to produce aberration corrected beamformer delays.

In FIG. 17 (similar to FIG. 2 of U.S. Pat. No. 6,682,487), a transducer probe 26 includes an array 10′ composed of elements 10e. The transducer array 10′ transmits and receives scanlines over an image field 14. Transducer probe 10 is a phased array probe which scans the image field by steering scanlines at different angles over the image field, although the invention is equally applicable to linear array and other transducer types. Transducer probe 26 is connected to a transmit beamformer 20 and a receive beamformer 25 by a transmit/receive switch 16 which protects the receive beamformer from high energy transmit pulses. Steering and focusing of the beams transmitted by the transmit beamformer is accomplished by transmit delay values provided by a beamformer delay generator 27, which also furnishes receive delay values to the receive beamformer to steer and dynamically focus received beams.

The beamformer delay generator may access a library of geometric focus delay tables which are provided to the beamformers for a desired beam angle and focus, or the delay generator may compute delay values for the beams prior to individual transmit-receive sequences. The transmit beamformer is responsive to a frequency control 23 which controls the frequency of the transmit waves, that is, either transmission at a fundamental frequency f or at a higher frequency nf.

The receive beamformer 25 forms coherent scanline data which is coupled to a detector 45. The detector may perform amplitude detection for B mode imaging or spectral detection for Doppler imaging. Detected echo signals are provided to an image processor 42 which processed the scanline signals into the desired image format. The resultant image is displayed on a display 100.

The receive beamformer 25 is also coupled to a harmonic echo data memory 32 and a non-harmonic echo data memory 34. When the receive beamformer is receiving harmonic echo data 2f in response to fundamental frequency f transmission, the harmonic image scanlines are stored in harmonic memory 32. When the receive beamformer is receiving fundamental frequency data 2f from fundamental frequency 2f transmission the fundamental image scanlines are stored in non-harmonic memory 34. If aberration correction is only to be performed at a discrete range r in the image field the memory size may be minimized by only gating echo data at the range r to the memories 32 and 34, which may be different storage areas of a single memory device.

Aberration correction may be done for a particular image range r where the scatterers to be imaged are located such as the transmit focal range, or for a plurality of different ranges. The harmonic and non-harmonic image data is provided to an aberration correction processor 30 which equalizes the harmonic and non-harmonic data for known systemic differences and compares the non-harmonic data to the harmonic data to estimate aberration correction values. The comparison is done by back propagation and comparison of the harmonic and non-harmonic data sets as more fully described below. The aberration correction values are coupled to the beamformer delay generator 27 where they are combined with the geometric delay values for the production of aberration-reduced ultrasonic images.

FIG. 18 (similar to FIG. 3 of U.S. Pat. No. 6,682,487) illustrates the aberration correction subsystem of the ultrasound system of FIG. 17 in greater detail. When a broadband transducer probe or scanhead is used for imaging, the receive beamformer, absent any filtering, will produce broadband scanline data. The harmonic used is the second harmonic, which is necessary to extract from the broadband signal. This is accomplished by a filter 46 set to pass the harmonic frequency 2f. The filter 47 may also separate harmonic signals from the fundamental signals by pulse inversion processing, as described in U.S. Pat. No. 5,951,478. The scanlines of the second harmonic image f_{2H} are stored in memory 32. When the scanlines at the fundamental frequency 2f are received they may be passed by the 2f filter 47 or the filter may be bypassed. The non-harmonic image scanlines are then stored in the 2f memory 34. The high frequency signals from the filter 47 are demodulated by mixing them to a baseband frequency range to reduce the amount of data and hence the bandwidth required for the aberration correction subsystem.

The harmonic and non-harmonic data sets are equalized for systemic differences by an f_{2H}:2f equalization processor 62. It is desirable for the two data sets to have the same equivalent aperture and bandwidth, for one example. The round trip spectral response of the two data sets can be adjusted to be identical, for another example. In the latter case more transmit pulses can be used at the higher transmit frequency 2f of the non-harmonic data or compensating receive filter adjustments can be made. In the former case the same aperture width can be used for the two data sets. To make a fundamental image look like a harmonic image triangular aperture weighting is used during fundamental imaging with a 1D array, or pyramidal weighting for the square or rectangular aperture of a 2D array. If transmit apodization is not available, the apodization weighting is applied to the back propagated receive data.

The equalized data sets are back propagated by Fourier transformation of each data set by a Fourier transform processor 64. The purpose of back propagation is to make a comparison of signals in the aperture domain. It is desirable to estimate aberration effects in the aperture domain, as this is the domain in which the corrections can be applied (e.g., gain and/or phase adjustment of each element in the active aperture). The changes in waveform amplitude and shape which were produced by propagation of the aberration time-shifted wave front to the receiving aperture may be removed by the applied corrections. Back propagation is done by using a Fourier transform applied across the image angles, that is, the differently steered scanlines of each data set.

The results of the Fourier transform are compared by a phase comparison or an amplitude comparison of the two results as by a division of the non-harmonic and harmonic results by an aperture ratio calculator 66. The comparison can be done by taking a ratio of the baseband signals, using a phase detector, taking a ratio of amplitude-demodulated signals, or a cross-correlation of the two signals. The complex ratio of the back-propagated data sets gives measured aberration correction values for both gain and phase correction.

While the mathematical analysis given at the outset of this specification was for reception by a single transducer element, a single element is non-selective as to region. The use of a group of receive elements as a subarray enables the use of steered and focused beams and the estimation of different aberration values for different regions of tissue, as well as improved signal-to-noise. This is done by steering a receive subarray to an image region for which aberration data is to be obtained. Transmission is now done over a range of transmit steering angles to fully cover the region within the receive beam profile. The receive steering angle is held fixed.

Harmonic and non-harmonic image data is acquired in this manner and stored for processing to generate the aberration correction values for the region. The use of a subarray of elements also fits well with subarray beamforming often used for three-dimensional imaging with a 2D array, as shown in U.S. Pat. Nos. 5,229,933 and 5,997,479. A separate aberration correction value may be estimated for each group of elements and the size of a group using the same value may be selected to correspond to the aberrator size. The range of steering angles over which the same correction value is used may be selected, as well as the range of elements which use the same correction value. The two are related, as a larger range of steering angles corresponds to a smaller number of transducer elements.

In a 2D array embodiment to provide aberration correction during three-dimensional imaging, a 2D array of 48 elements by 60 elements may be divided into an N×M array of 12 groups by 12 groups. This results in each group being a 4 by 5 group of 20 elements. When the subsystem of FIG. 18 is used for aberration correction, 12×12 or 144 2D Fourier transforms are calculated for each data set. The Fourier transforms can be done over 20 steering angles, 4 in elevation and 5 in azimuth.

While it is possible to do the transmission and reception of both harmonic and non-harmonic data sets at the beginning of every image frame, if neither data set is used for the image frame, the frame rate will be reduced to a third of the frame rate without compensation. To avoid this frame rate reduction the imaged data set can be used for one of the aberration correction data sets. The acquisition of the data sets can be done on a periodic basis such as once every ten image frames. The acquisition and correction can be done on a time-interleaved basis, where the acquisition and correction of only one or a few subarrays or for one or a few ranges can be performed between image frames. Over the course of a number of image frames the entire array and image field will be corrected and then updated.

Commonly owned U.S. Pat. No. 6,508,764 (the '764 patent), incorporated by reference herein, describes an ultrasound system and method for aberration correction processing in conjunction with finely pitched transducer elements and/or aberration correction processing in conjunction with a hierarchical control scheme. More particularly, the '764 patent teaches aberration correction and detection in conjunction with the beamforming operation occurring in the main ultrasound system, the results of which aberration correction and detection are provided by the beamformer to control transmit and receive beamforming.

The present invention combines the benefits of aberration correction with the benefits of microbeamforming in an ultrasound diagnostic imaging system, such that both partial beamforming (in the microbeamformer) and at least some part of the phase aberration detection correction processes are accomplished in the transducer probe proximate the transducer array. Accordingly, aberration correction applied at or proximate the microbeamformer results in a significant simplification of the overall aberration correction technique for the entire ultrasound diagnostic imaging system.

In one embodiment, the invention consists of an aberration correction detector which detects aberration corresponding to individual transducer elements. The aberration correction is organized according to sub-groups or sub-arrays of transducer elements. An aberration detector either in the transducer probe or in the main beamformer in communication with the main beamformer therein detects aberration and provides corresponding aberration values to an aberration correction processor, typically in the base or main system. The aberration processor, in conjunction with the main beamformer, provides correction values to the transducer probe in accordance with the sub-grouping arrangement implemented by the main beamformer and microbeamformer. The correction values per sub-groupings are sent to the microbeamformer either directly from the aberration processor or from an aberration compensation correction circuit, proximate to or within the microbeamformer circuitry.

More particularly, the microbeamformer and aberration correction circuitry is disposed within a transducer probe to realize the aforementioned benefits of carrying out phase aberration correction with the microbeamforming operation in the transducer probe. The base ultrasound system is disposed in a separate housing (from that of the transducer probe) and is in communication with the probe (or scanhead) by cable or wireless connection. The aberration processor in the base ultrasound system may detect aberration in conjunction with the main beamformer, and calculates correction factors by sub-group or sub-array of transducer elements, or may receive detected aberration values from an aberration detector in the probe. The aberration processor generates correction values by sub-group, which correction values may be provided directly from the aberration processor to the microbeamformer, or as mentioned, indirectly through a compensation correction circuit, which may be part of or separate from the microbeamformer. Alternatively, the correction values may be calculated and provided to the microbeamformer through the main beamformer, or to the microbeamformer via compensation correction circuitry in the probe to implement the aberration correction within the microbeamforming operations in accordance hereto.

FIG. 19 is a block diagram of a base ultrasound system 41 and transducer probe 26′ by which the present invention may be practiced. It will be appreciated by those skilled in the art that the ultrasound imaging system 41 and transducer probe 26′ does not show every element required for operation of ultrasound imaging and processing, but shows only those elements required to generally represent an ultrasound imaging system and to provide a framework for detailed discussion of the present invention. Those of ordinary skill in the art will recognize the applicability of the present invention to a wide variety of ultrasound systems which may differ significantly from those shown in any figure herein.

Transducer probe 26′ includes transducer array 10″, constructed so that each element of the transducer array is electrically connected to a microbeamformer 37 for microbeamforming control. Microbeamformer 37 is connected via cable to a main beamformer 39 and aberration processor 43 in ultrasound system 41. In the embodiment shown (for exemplary purposes only), the microbeamformer includes compensation correction circuitry 45 which implements the aberration correction factors, delivered from the main beamformer, to individual transducer elements. The compensation correction circuitry 45 may also include aberration detection circuitry for detecting aberration per transducer element, for transfer to the beamformer and/or aberration processor. However, the actual aberration detection may be implemented, not in the microbeamformer, but in the main beamformer circuitry, with or without the help of aberration processor 43, or directly at the circuitry by which the aberration processing can be carried out in the base ultrasound system. The aberration compensation correction circuitry 45 must be in communication with at least the microbeamformer 37 to synchronize its correction with the line sequence of elements being activated.

More particularly, the present invention, in any embodiment, incorporates an aberration correction process in concert with the microbeamforming operation. The aberration correction process may be any known aberration process known in the art as long as it is implemented to a greater or lesser or extent within the transducer probe in conjunction with the microbeamformer. In this manner, the main beamformer in the base ultrasound system, with or without a separate aberration processor, may be utilized for other operations, and the electrical connections to the ultrasound transducer may be kept to a minimum while effectively controlling the beamforming and aberration correction processes.

In another exemplary embodiment, FIG. 20 shows both aberration detection circuitry 43 and aberration correction circuitry 45 located within the transducer probe 26″ as separate circuits from the microbeamformer 37. The microbeamformer is connected directly to the 2-D (or multidimensional matrix) 10″. Of course as mentioned above, each of the elements comprising array 10″ are differentiated by sub-arrays or sub-groups in accordance with microbeamforming. FIG. 2 of prior art U.S. Pat. No. 6,508,764, and associated text therein, provides a good description of the operation of sub-groups and operation thereof. But as distinguished from the sub-groups and their operation as described in the prior art patent, the transducer elements and microbeamformer of this invention are combined in the transducer probe to perform both aberration detection and correction in cooperation with the microbeamformer. Again, the circuitry required to implement same, as known to those skilled in the art, may be part of the microbeamformer circuitry or implemented in the probe as separate circuit elements. The inventions as described are particularly useful in live 3D operation, where the time freed by the main beamformer in carrying out the functions of aberration detection and correction, performed in conjunction with the microbeamforming operation, may be used for other processes. That is, the hierarchical configuration allows precise control over a multitude of transducer elements while reducing the number of leads required to connect a multidimensional, e.g., 2-D matrix array, to the main or base ultrasound system. The same hierarchical configuration allows for aberration correction algorithms to be applied to each subgroup, where the bulk of the aberration detection and correction processes is implemented by sub-group in the transducer probe in conjunction with the microbeamforming processes.

Although only a few exemplary embodiments have been described in detail above, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

1. An ultrasonic diagnostic imaging system, comprising:
a base ultrasound imaging system including image processing circuitry,
a transducer probe connected to the base ultrasound imaging system housing via a cable, and
a display coupled to the image processing circuitry including a main beamformer within the base ultrasound imaging system for displaying ultrasound images;
wherein the transducer probe includes a multidimensional array of transducer elements, which elements transmit beams of ultrasonic energy into a volumetric region and receive ultrasound signals in return, a microbeamformer coupled to the array of transducer elements and to the main beamformer, which microbeamformer implements a partial beamforming, an aberration detection and an aberration correction function, and the main beamformer directs the microbeamformer to correct for aberration and to drive the transducer elements by sub-group, which main beamformer is responsive to the partially beamformed ultrasound signals.

2. The ultrasound diagnostic imaging system as set forth in claim 1, wherein the transducer probe further includes aberration detection circuitry one of connected to and incorporated within the microbeamformer.

3. The ultrasound diagnostic imaging system set forth in claim 1, further including compensation correction circuitry one of electrically connected to and incorporated within the microbeamformer for implementing aberration correction values for the transducer elements.

4. The ultrasound diagnostic imaging system as set forth in claim 1, further including an aberration processor in the base ultrasound system coupled to at least the main beamformer for calculating aberration correction factors based on a sub-grouping of transducer elements.

5. The diagnostic imaging system as set forth in claim 4, wherein the aberration processor includes aberration detection circuitry.

6. The diagnostic imaging system as set forth in claim 4, wherein the microbeamformer includes aberration compensation circuitry, the microbeamformer is coupled to al least one of the main beamformer and aberration processor, and receives correction values generated in the aberration processor, by sub-grouping, and implementation by the microbeamformer.

7. The three dimensional ultrasonic diagnostic imaging system set forth in claim 1, where the main beamformer spatially samples the volumetric region in one of a triangular and hexagonal pattern.

8. The three dimensional ultrasonic diagnostic imaging system of claim 1, wherein the main beamformer comprises a multiline beamformer which produces a plurality of scanlines for every transmit beam.

9. The three dimensional ultrasonic diagnostic imaging system of claim 6, wherein the ultrasound system further includes an interpolator responsive to the partially beamformed ultrasound signals which forms interpolated scanlines.

10. The three dimensional ultrasonic diagnostic imaging system of claim 1, wherein elements of the multidimensional array are grouped in hexagonally shaped patches, corresponding to sub-groups; and
wherein the elements of each sub-group or patch are coupled to the microbeamformer, which acts to beamform signals received by the sub-group or patch.

11. An ultrasonic diagnostic imaging system including a base system in a housing and an ultrasonic transducer probe communicatively coupled to the base system, which ultrasound diagnostic imaging system corrects for speed of sound aberration and implements a microbeamforming operation within the ultrasonic transducer probe, wherein the ultrasonic transducer probe comprises:
a multidimensional transducer array for transmitting and receiving ultrasonic waves; and
a microbeamformer coupled to the transducer array and capable of causing the transducer elements comprising the transducer array to transmit and receive ultrasonic waves at a plurality of selectable frequencies; and
wherein the base system further includes a main beamformer delay generator coupled to at least one of the main beamformer and the microbeamformer in the transducer probe for providing geometrically derived delays to the microbeamformer according to transducer element sub-groupings;
a data storage device coupled to the main beamformer which acts to store an aberration correction data set; and
an aberration correction processor, responsive to the aberration correction data set and having an output coupled to at least one of the main beamformer delay generator and the microbeamformer, which processor provides the at least one of the microbeamformer and the main beamformer delay generator with aberration correction values based on the aberration correction data set.

12. A method of compensating for aberrations as part of a microbeamforming operation within an ultrasound diagnostic imaging system which includes a main system housing and a separate ultrasound transducer probe, comprising the steps of:
grouping elements of a multidimensional transducer array, which is disposed in the transducer array, into subgroups;
implementing a microbeamforming operation on the subgroups of the multidimensional transducer array while transmitting and receiving ultrasound energy to implement ultrasound imaging;
detecting aberration in signals output by each of the subgroups of the transducer array;
compensating for aberration in the signals output by each subgroup by generating a compensation value for each subgroup, interpolating a compensation value for each element in a subgroup and using the interpolated compensation values to adjust a signal derived from the output of each element at the transducer probe.
